(19) United States
(12) Patent Application Publication
(10) Pub. No.: US 2012/0189653 A1
(43) Pub. Date: Jul. 26, 2012

(54) FOOD/BEVERAGE AND PHARMACEUTICAL COMPOSITION FOR ORAL ADMINISTRATION FOR IMPROVEMENT IN ACIDIC URINE EACH COMPRISING FUCOIDAN AS ACTIVE INGREDIENT

(75) Inventors: Ichiro HISATOME, Yonago-shi (JP); Yasuaki SHIRAYOSHI, Yonago-shi (JP); Hiroyuki TAKEYA, Yonago-shi (JP); Ryota TESHIMA, Yonago-shi (JP); Yasunari MIKI, Sakaiminato-shi (JP)

(73) Assignees: Marine Products Kimuraya Co., LTD., Sakaiminato-shi (JP); Tottori University, Tottori-shi (JP)

(21) Appl. No.: 13/439,936

(22) Filed: Apr. 5, 2012

Related U.S. Application Data
(62) Division of application No. 12/530,332, filed on Sep. 8, 2009.

(30) Foreign Application Priority Data
Mar. 27, 2007 (JP) 2007-081453
Jan. 4, 2008 (JP) 2008-000088

(51) Int. Cl. A61K 31/737; A61P 13/02

(52) U.S. Cl. 424/195.17; 514/54

(57) ABSTRACT

The present invention provides a food/drink or pharmaceutical composition for oral administration for improving an acidic urine, comprising fucoidan or a fucoidan-containing material as an active ingredient, characterized in that a urinal pH is persistently increased; use of fucoidan or a fucoidan-containing material for production of the food/drink or the pharmaceutical composition for oral administration; and a method for improving an acidic urine by persistently increasing a urinal pH of a patient, which comprises orally administering fucoidan or a fucoidan-containing material to a patient.

### CROSS REFERENCE TO RELATED APPLICATIONS

This application is a divisional of U.S. application Ser. No. 12/530,332 filed on Sep. 8, 2009, which is a National Stage of PCT/JP2008/055525 filed on Mar. 25, 2008 and which claims priority to Japanese Application Serial No. 2007-081453 filed on Mar. 27, 2007 and Japanese Application Serial No. 2008-000088 filed on Jan. 4, 2008, the entire contents of which are incorporated by reference herein.

### TECHNICAL FIELD

The present invention relates to a food/drink and a pharmaceutical composition for oral administration, which are used for improving an acidic urine. More particularly, the present invention relates to a food/drink for improving an acidic urine containing fucoidan or a fucoidan-containing material as an active ingredient, in which the acidic urine improving effect lasts for a long period of time. The present invention also relates to a pharmaceutical composition for oral administration used for improving an acidic urine, containing fucoidan or a fucoidan-containing material as an active ingredient, in which the pH of a urine is persistently increased. Further, the present invention relates to use of fucoidan or a fucoidan-containing material for production of the food/drink and the pharmaceutical composition for oral administration.

### BACKGROUND ART

An acidic urine is generated in a patient of metabolic syndrome in which hypertension, hyperlithuria, hyperlipemia, glucose tolerance abnormality and obesity are complicated, and has a great medical problem that kidney function disorder such as urinary caleulus and proteinuria is generated. As a drug for treating/improving an acidic urine, a urinal alkalinizing agent such as a sodium/potassium citrate preparation, and sodium bicarbonate have hitherto been used (see Non-Patent Document 1, etc.). However, since dosing becomes difficult due to the reason that a tablet has a large size, there is a high possibility that treatment is interrupted. Therefore, it has been required to develop an acidic urine improving drug, which has a small burden on a patient and the effect of which lasts for a long period of time.

[Non-Patent Document 1] DRUGS IN JAPAN, THERAPEUTIC DRUNGS, 2006 edition, page 678

### DISCLOSURE OF THE INVENTION

### Problems to be Solved by the Invention

The problem to be solved by the present invention was to develop an acidic urine improving drug which has a small burden on a patient, and the effect of which lasts for a long period of time.

### Means for Solving the Problems

In order to solve the aforementioned problem, the present inventors intensively studied and found that the problem can be solved by using a food/drink or a pharmaceutical composition for oral administration, containing fucoidan or a fucoidan-containing material as an active ingredient. Thus, the present invention has been completed.

That is, the present invention provides:
(1) a food/drink for improving an acidic urine, comprising fucoidan or a fucoidan-containing material as an active ingredient, characterized in that a urinal pH is persistently increased;
(2) the food/drink according to (1), wherein a urinal pH is increased by 0.2 or more;
(3) the food/drink according to (1) or (2), wherein a urinal pH is increased over 24 hours or longer;
(4) the food/drink according to any one of (1) to (3), wherein 600 mg or more of fucoidan is taken per day;
(5) the food/drink according to any one of (1) to (4), wherein the fucoidan-containing material is mozuku or an extract thereof;
(6) the food/drink according to any one of (1) to (5), which is a supplement;
(7) use of fucoidan or a fucoidan-containing material for production of a food/drink for improving an acidic urine, characterized in that a urinal pH is persistently increased;
(8) a pharmaceutical composition for oral administration for improving an acidic urine, comprising fucoidan or a fucoidan-containing material as an active ingredient, characterized in that a urinal pH is persistently increased;
(9) the pharmaceutical composition according to (8), wherein a urinal pH is increased by 0.2 or more;
(10) the pharmaceutical composition accosting to (8) or (9), wherein a urinal pH is increased over 24 hours or longer;
(11) the pharmaceutical composition according to any one of (8) to (10), wherein 600 mg or more of fucoidan is taken per day;
(12) the pharmaceutical composition according to any one of (8) to (11), wherein the fucoidan-containing material is mozuku or an extract thereof;
(13) use of fucoidan or a fucoidan-containing material for production of an oral administration drug for improving an acidic urine, characterized in that a urinal pH is persistently increased;
(14) the food/drink according to (13), wherein a urinal pH is increased by 0.2 or more;
(15) the food/drink according to (13) or (14), wherein a urinal pH is increased over 24 hours or longer;
(16) the food/drink according to any one of (13) to (15), wherein 600 mg or more of fucoidan is taken per day;
(17) the food/drink according to any one of (13) to (16), wherein the fucoidan-containing material is mozuku or an extract thereof;
(18) a method for improving an acidic urine by persistently increasing a urinal pH of a patient, which comprises orally administering fucoidan or a fucoidan-containing material to a patient.
(19) the food/drink according to (18), wherein a urinal pH is increased by 0.2 or more;
(20) the food/drink according to (18) or (19), wherein a urinal pH is increased over 24 hours or longer;
(21) the food/drink according to any one of (18) to (20), wherein 600 mg or more of fucoidan is taken per day; and
(22) the food/drink according to any one of (18) to (21), wherein the fucoidan-containing material is mozuku or an extract thereof.

### Effects of the Invention

According to the present invention, there are provided a food/drink and a pharmaceutical composition for oral administration, which are used for improving an acidic urine, characterized in that a pH of a urine is persistently increased. Moreover, the food/drink and the pharmaceutical composition for oral administration of the present invention are easily taken by a patient. Therefore, a burden on a patient is small and an acidic urine can be improved over a long period of time.

### BRIEF DESCRIPTION OF THE DRAWINGS

A left panel of FIG. 1 is a graph showing a change in a urinal pH before and after administration of fucoidan three times per day. A lower solid line shows a urinal pH before administration of fucoidan, and an upper solid line shows a urinal pH after administration of fucoidan. The symbol * shows p<0.05, significant. A right panel of FIG. 1 is a circle graph showing an efficacy rate of fucoidan administration (half circle 50%, full circle 100%). Gray shows effective, and black shows no effect.

FIG. 2 is a graph showing a change in a urinal pH before and after eating of mozuku. A lower solid line shows a urinal pH before eating of mozuku, and an upper solid line shows a urinal pH after eating of mozuku.

FIG. 3 is a graph showing a change in a urinal pH before and after administration of a citrate preparation once per day. A lower solid line shows a urinal pH before administration of a citrate preparation, and an upper solid line shows a urinal pH after administration of a citrate preparation. The symbol * shows p<0.05, significant.

### BEST MODE FOR CARRYING OUT THE INVENTION

The present invention, in one aspect, provides a food/drink for improving an acidic urine, comprising fucoidan or a fucoidan-containing material as an active ingredient, characterized in that a pH of a urine is persistently increased. Fucoidan is a sulfur-containing polysaccharide found in the natural world, and is contained in a seaweed such as mozuku (Nemacystus decipiens) or wakame (Undaria pinnatifida) in a large amount. In recent years, it has been found that fucoidan serves in promotion of regeneration of a tissue, adjustment of immunological balance, and suicide of a cancer cell. However, it has not been scientifically demonstrated that an acidic urine is improved by shifting a urine to an alkaline side persistently over a long period of time, thereby, generated various diseases are prevented or improved, like the present invention.

Fucoidan which is an active ingredient in the food/drink may be a purified product or a crude purified product, for example, an extract from seaweed such as mozuku. The fucoidan-containing material as an active ingredient in the food/drink of the present invention may be any material as long as it contains fucoidan and is non-toxic to a human. Examples of preferable fucoidan-containing material in the present invention include seaweed, particularly, brown algae. Examples of the fucoidan-containing brown algae include mozuku (Okinawa-mozuku (Cladosiphon okamuranus), thread-form mozuku), wakame (Undaria pinnatifida), Eisenia bicyclis, Kjellamaniella crassifolia Miyabe, Laminaria Japonica Areschoug, Ecklonia kurome, Ecklonia cava Kjellman, Laminaria angustata, Sargassum siliquastrum, Sargassum fusiforme. Sargassum fulvellum, Sargassum patens C. Agardh, Sargassum humeri, Bladderwrack and Sargassum thumbergii, being not limiting. The “mozuku” as used herein includes both of Okinawa-mozuku and thread-form mozuku.

The food/drink of the present invention is characterized in that the effect of increasing a pH of a urine lasts for a long period of time. For example, a patient may take the food/drink of the present invention three times a day, twice a day, or once a day. Alternatively, for example, a patient may take the food/drink of the present invention at every meal, or before or after the meal, or a patient may take the food/drink once a day, for example, at breakfast, or before or after thereof, at lunch, or before or after thereof, at dinner, or before or after thereof. Alternatively, the food/drink of the present invention may be taken between meals.

Increase in a urinal pH due to intake of the food/drink of the present invention is preferably the pH of about 0.2 or more over the case of intake of no food or drink of the present invention. In order not to crystallize a component in a urine, it is preferable to keep the pH of a urine of a patient 6.0 or higher.

In order to obtain the above effect, an amount (dry weight) of fucoidan taken in the food/drink of the present invention is preferably about 600 mg or more per day in the case of an adult.

Since fucoidan as such is tasteless and odorless, a variety of foods or drinks can be produced without influencing on a flavor. Alternatively, a food/drink utilizing a flavor of the fucoidan-containing material may be produced. For example, a miso soup or a soup with a fucoidan or a fucoidan extract from mozuku added thereto may be produced. A fucoidan powder, or a fucoidan extract from mozuku, or dry mozuku may be added to a powdery or pasty miso soup or soup stock to produce an instant miso soup or soup. Alternatively, for example, a fucoidan extract from mozuku is concentrated and filled into a pack, or is treated with lyophilization to formulate into a powder or a granule, and is subjected to treatment such as filling into a suitable wrapping or container, thereby, a form by which a user itself add to an arbitrary food or drink, and take it may be obtained. Examples of the food/drink using fucoidan or the fucoidan-containing material include sauce, ketchup, soy sauce, miso, dressing, broth, soup, dried seasoning powder, stock green tea pickle, various drinks (health drink, juice, carbonated drink, refreshing drink, coffee drink, tea drink, milk drink, lactic acid bacteria drink, powder drink etc.), biscuit, cookie, cake, snack, rice cracker, and bread, being not limiting. In addition, examples of the form of the food/drink containing fucoidan or a fucoidan-containing material include tablets and capsules.

Furthermore, the food/drink of the present invention may be a supplement containing fucoidan or a fucoidan-containing material. The supplement can be formulated into a form such as tablets, capsules, granules, and powders by the method known to a person skilled in the art. Like this, when the food/drink of the present invention is used, a user or a patient can routinely take fucoidan without any resistance, and can improve an acidic urine over a long period of time. Alternatively, the therapeutic effect can be also promoted by joint intake by a person who has already undergone a treatment of an acidic urine.

The present invention, in another aspect, provides a pharmaceutical composition for oral administration used for improving an acidic urine comprising fucoidan or a fucoidan-containing material as an active ingredient, characterized in that a pH of a urine is persistently increased. The pharmaceutical composition for oral administration of the present invention can be formulated into various oral dosage forms such as concentrated solutions, powders, granules, tablets, capsules and drinks. A process for producing these dosage forms is known, and a process such as mixing, dissolution, grinding, compression, drying and the like can be appropriately used. In addition, the pharmaceutical composition for oral administration of the present invention can be administered as it is, or can be administered by arbitrarily adding to, for example, miso soup, green tea, or other foods or drinks.

The pharmaceutical composition for oral administration of the present invention is characterized in that the effect of increasing a urinal pH lasts for a long period of time. For example, the pharmaceutical composition for oral administration of the present invention may be administered to a patient three times a day, twice a day, or once a day. Alternatively, for example, the pharmaceutical composition for oral administration of present invention may be administered to a patient at every meal, or before or after thereof, or the pharmaceutical composition for oral administration of the present invention may be administered to a patient once per day, for example, at breakfast, or before or after thereof, at lunch, or before or after thereof, at dinner, or before or after thereof. Alternatively, the pharmaceutical composition for oral administration of the present invention may be administered between meals.

It is preferable that increase in a urinal pH due to administration of the pharmaceutical composition for oral administration of present invention is the pH of about 0.2 or more over the case of administration of no pharmaceutical composition for oral administration of the present invention. In order not to crystallize a component in a urine, the pH of the urine of a patient is preferably kept at 6.0 or higher.

In order to obtain the above effect, an amount (dry weight) of fucoidan administered in the pharmaceutical composition for oral administration of present invention is preferably about 600 mg or more per day in the case of an adult.

In the pharmaceutical composition for oral administration of the present invention, not only fucoidan but also one or more kinds of other active ingredients may be mixed. For example, a component which increases the pH of the urine, such as a sodium/potassium citrate preparation, sodium bicarbonate or the like may be used jointly in the pharmaceutical composition for oral administration of the present invention. Alternatively, the therapeutic effect can be also promoted by joint intake of the pharmaceutical composition for oral administration of the present invention by a person which has already undergone a treatment of an acidic urine.

The present invention, in another aspect, provides use of fucoidan or a fucoidan-containing material for production of a food/drink or a pharmaceutical composition for oral administration for improving an acidic urine, characterized in that a pH of a urine is persistently increased. The fucoidan-containing material is as described above. A used amount of fucoidan or the fucoidan-containing material is preferably such an amount that an adult can take 600 mg or more of fucoidan (dry weight) per day. The food/drink and the pharmaceutical composition for oral administration obtained by using the present invention are routinely accepted by a patient, and an acidic urine can be improved over a long period of time.

Furthermore, the present invention provides a method of persistently increasing a urinal pH of a patient to improve an acidic urine, comprising orally administering fucoidan or a fucoidan-containing material to a patient.

The present invention will be described in more detail or specifically by way of Examples, but the Examples should not be construed to limit the present invention.

### EXAMPLE 1

Urinal pH increasing effect of administration of fucoidan or fucoidan-containing material.

Fucoidan was administered to 11 hypertension patients at breakfast (seven o'clock), at lunch (twelve o'clock) and at dinner (nineteenth o'clock) continuously for 7 days. Fucoidan was a form of a liquid extract from mozuku. This was appropriately added to miso soup, green tea, or other food at 10 ml (containing about 200 mg of fucoidan in terms of a dry weight) per one time, followed by administration. Immediately before breakfast, a urine was collected, and a urinal pH was investigated using a test paper which can measure a pH. Results are shown in FIG. 1. A lower solid line of FIG. 1 is an average urinal pH before breakfast, before lunch, or before dinner on the previous day of fucoidan administration. An upper solid line of FIG. 1 is an average urinal pH before breakfast, before lunch, or before dinner on 7 day from initiation of administration of fucoidan. As seen from FIG. 1, as compared with a urinary pH before breakfast, before lunch, before dinner on the previous day from fucoidan administration, a urinary pH was significantly shifted to alkaline after fucoidan administration, and a shift width was about 0.2 pH or more. Depending on a patient, a urinal pH before breakfast was 5.24 before fucoidan administration, and the pH was increased to 6.02 after fucoidan administration. In addition, an efficacy rate (ratio of patients having increased urinal pH) of fucoidan administration was 60 to 90% throughout before breakfast, before lunch and before dinner, and an efficacy rate before breakfast was particularly high. This shows that the urinal pH increasing activity of fucoidan lasted for a long period of time. Since urinary caleulus or the like is easily generated at night, it is worthy of special mention that an efficacy rate before breakfast is high.

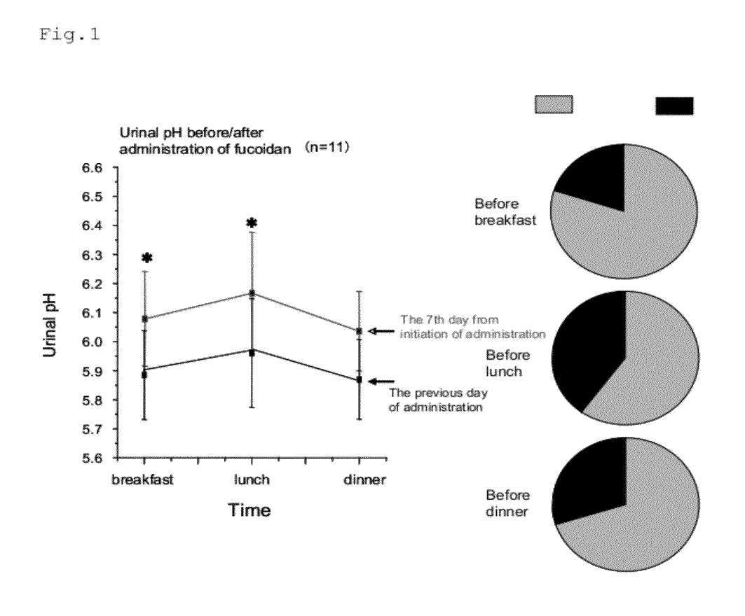

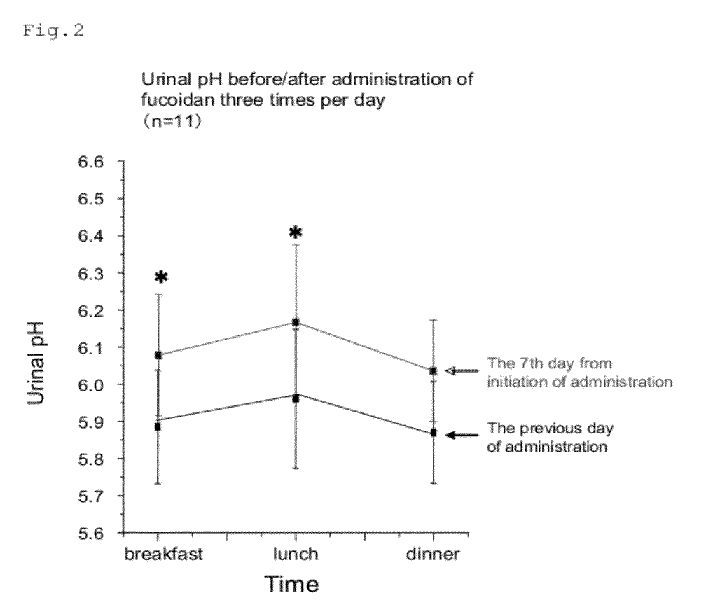

Further, mozuku containing much fucoidan was made to be eaten by 11 hypertension patients, and change in a urinal pH was investigated. At breakfast, about 120 g of mozuku (mozuku about 60 g sanbaizu (mixture of vinegar, soy sauce and sugar) about 60 g) was eaten (taken once a day). One time mozuku contained about 600 mg of fucoidan in terms of dry weight). Before breakfast, before lunch and before dinner, a urine was collected, and a urinal pH was investigated using a test paper which can measure the pH. Results as shown in FIG. 2. A urinal pH before breakfast, before lunch and before dinner on the previous day of eating of mozuku is shown in a lower solid line, and a urinal pH before breakfast, before lunch and before dinner on 7 day from initiation of eating mozuku is shown in an upper solid line. As compared with before eating of mozuku, a urinal pH was significantly shifted to alkaline after eating of mozuku, and a shift width was about 0.2 pH or more. Moreover, it was seen that the urinal pH increasing effect lasts for at least one day (24 hours).

### COMPARATIVE EXAMPLE 1

### Urinal pH Increasing Effect of Administration of Sodium/Potassium Citrate reparation

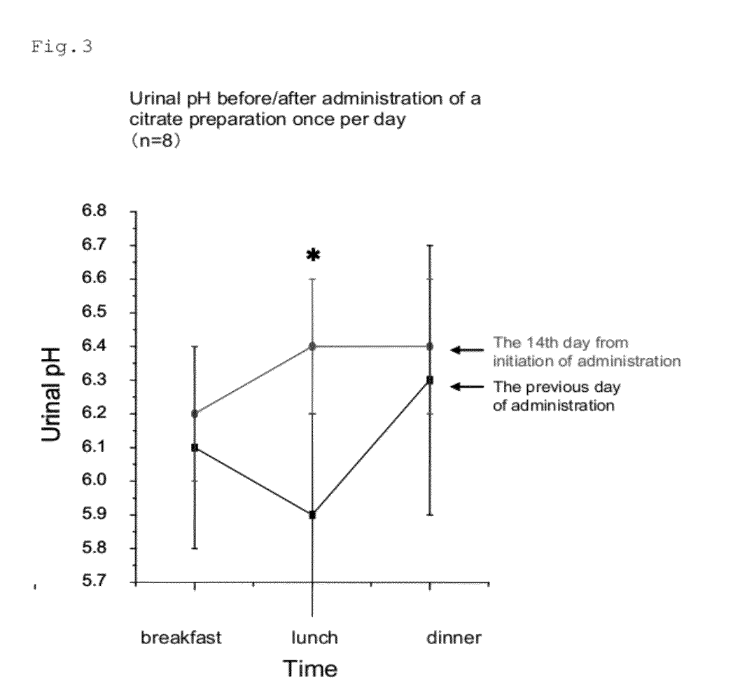

One gram of a sodium/potassium citrate preparation was administered to 8 hypertension patients at breakfast (seven o'clock), at lunch (twelve o'clock) and at dinner (nineteenth o'clock) continuously for 14 days (administration once per day). A urine immediately before a meal was collected, and a urinal pH was investigated using a test paper which can measure the pH. The results are shown in FIG. 3. A lower solid line of FIG. 3 is an average urinal pH before breakfast, before lunch, and before dinner on the previous day of administration of a sodium/potassium citrate preparation. An upper solid line of FIG. 3 is an average urinal pH before breakfast, before lunch and before dinner on 14 days from initiation of administration of a sodium or potassium citrate preparation.

By comparing the urinal pH increasing effect of administration of a sodium/potassium citrate preparation which is the previous urine alkalinizing drug (FIG. 3) with urinal increasing effect of administration of the fucoidan-containing material (FIG. 1), it was found that the alkalinizing activity of fucoidan has the effect equivalent to or superior to that of administration of urine alkalization activity of a sodium/potassium citrate preparation. In addition, by administering fucoidan later to the case of preceding administration of a sodium/potassium citrate preparation, it was seen that a urinal pH can be further alkalinized, and it was also confirmed that the urine alkalizing activity of fucoidan is independent of the activity of a sodium/potassium citrate preparation.

### INDUSTRIAL APPLICABILITY

The present invention can be utilized in the field of foods, medicaments and the like.

1. A method for treating acidic urine, comprising administering to a patient in need thereof an effective amount of fucoidan or a fucoidan-containing material.

2. The method of claim 1, wherein the fucoidan or fucoidan-containing material is orally administered.

3. The method of claim 2, wherein the fucoidan or fucoidan-containing material is in the form of a pharmaceutical composition, a drink, a food, or a food supplement.

4. The method of claim 1, wherein the treatment results in an increase in the pH of the patient's urine.

5. The method of claim 4, wherein the increase is a persistent increase.

6. The method of claim 4, wherein the pH of the patient's urine is increased by 0.2 or more.

7. The method of claim 4, wherein the pH of the patient's urine is increased over a period of 24 hours or longer.

8. The method of claim 1, wherein the treatment results in a patient urine pH of 6.0 or higher.

9. The method of claim 1, wherein the amount of fucoidan administered is 600 mg or more per day.

10. The method of claim 1, wherein the fucoidan-containing material is a seaweed or an extract thereof.

11. The method of claim 10, wherein the seaweed is a brown algae.

12. The method of claim 11, wherein the brown algae is mozuku or wakame.

13. The method of claim 12, wherein the brown algae is mozuku.
